(19) United States
(12) Patent Application Publication
(10) Pub. No.: US 2010/0006665 A1
(43) Pub. Date: Jan. 14, 2010

(54) Aqueous Biocidal Composition, and Methods and Devices for Dispersing It

(76) Inventor: Keith Seymour, Marseyside (GB)

Correspondence Address: RENNER KENNER GREIVE BOBAK TAYLOR & WEBER, FIRST NATIONAL TOWER FOURTH FLOOR, 106 S. MAIN STREET, AKRON, OH, 44308, US

(21) Appl. No.: 12/084,869

(22) PCT Filed: Nov. 10, 2006
(86) PCT No.: PCT/GB2006/050384

§ 371 Date: Dec. 22, 2008

(30) Foreign Application Priority Data
Nov. 10, 2005 (GB) 0522906.7

(51) Int. Cl. A61L 9/04; A01N 33/12; A01N 43/80

(52) U.S. Cl. 239/6; 514/642; 514/372; 239/44

(57) ABSTRACT

An aqueous biocidal composition comprising, a nitrogen-containing biocide, a sulphur-containing biocide and an oleophilic carrier. A method and device for dispersing such a biocidal composition. A pouch for protecting produce that incorporates the biocidal composition.

### FIELD OF THE INVENTION

This invention relates to biocidal compositions, and methods of dispersal thereof.

### BACKGROUND TO THE INVENTION

In many enclosed spaces, particularly in enclosed rooms, it is desired to prevent the build-up of microbes in the air within the enclosed space. Microbes may be carried in the air within enclosed spaces by such vectors as air conditioning, air currents from open windows and the like. Examples of enclosed spaces in which it is desirable to reduce or remove microbes from air include cabins and quarters in aircraft, ships and cruise liners, hospital wards, nursing homes, clinics etc, bars and restaurants and gymnasiums, health spas and hotels. Each of these examples of enclosed spaces can very quickly become contaminated with microbes as people enter and exit the enclosed spaces, and through air conditioning and filtering systems.

It is particularly important to remove microbes in clinical environments such as hospitals, nursing homes and clinics, as the enclosed spaces within these buildings can quickly build up large quantities of microbes, which may then infect patients with the clinical environment, and add to existing medical complications. Indeed it is known in hospital wards that diseases such as Legionnaires disease are carried by air conditioning systems through the wards; and many other strains of bacterium whether airborne or static are known to exist in hospital wards, such as penicillin resistant bacteria, multi drug resistant Staphylococcus Aureus and the like.

In order to reduce or eliminate microbes, whether airborne or static, in enclosed habitable spaces, many devices are known. It is known to add anti-microbial compositions to water in air conditioning systems or to insert antimicrobial devices within air ducts in air conditioning systems in order that air passing through the ducts is contacted by anti-microbial compositions within the devices.

Furthermore, it is known to produce devices, which plug into the mains electricity, in which a wick or the like is heated to draw up and disperse, usually by evaporation, an anti-microbial composition, which is then released into the atmosphere in the enclosed space.

For known devices which disperse biocides into the atmosphere in enclosed spaces, there is consistently a problem in achieving a steady concentration of biocide in the atmosphere, which is effective to kill microbes, but does not build to a level dangerous to human health.

Mainly biocidal compositions for use in vaporisers are aqueous based compositions, in which the biocide disperses with the water from the composition, upon heating. It can be difficult to control the rate of evaporation, and to ensure that sufficient biocide disperses into the atmosphere with the water vapour. It would be advantageous to provide a biocidal composition which can be utilised in vaporisers, or other biocidal applicators, in which the biocides are substantially completely miscible with solvents and co-ingredients, and in which the biocides and solvent carrier can evaporate and disperse at optimal conditions to allow complete dispersal of the biocide in an enclosed atmosphere.

It is therefore an aim of preferred embodiments of the present invention to overcome or mitigate at least one problem of the prior art, whether disclosed hereinabove or not.

### SUMMARY OF THE INVENTION

According to a first aspect of the present invention, there is provided an aqueous biocidal composition comprising, a nitrogen-containing biocide, a sulphur-containing biocide and an oleophilic carrier.

The nitrogen-containing biocide may be a non-oxidising or oxidising nitrogen-containing biocide.

The nitrogen-containing biocide may comprise an amide biocide, a triazine, a guanidine, a quaternary ammonium compound, or mixtures or derivatives thereof, but is preferably a quaternary ammonium compound.

Suitably the quaternary ammonium compound comprises an optionally substituted linear or branched C₁-C₂₀ alkyl ammonium halide or alkylbenzylammonium halide. Preferably the alkylammonium halide is a dialkyldimethyl ammonium halide or an alkyldimethylbenzyl ammonium chloride.

The halide is preferably chloride, bromide or iodide, but is more preferably chloride.

Suitable quaternary ammonium compounds include dimethyl dibenzylammonium chloride, dodecyldimethyl ammonium chloride, ethylmethyl dibenzyl ammonium chloride and didecyl dimethyl ammonium chloride.

Suitably the nitrogen-containing biocide is present in the composition in an amount of at least 1wt % of the total weight of (the composition, preferably at least 1.5wt %, and more preferably at least 2wt %.

Suitably the nitrogen-containing biocide is present in the composition in an amount of no more than 10wt %, preferably no more than 7.5wt % and more preferably no more than 5wt %, of the total weight of the composition.

The sulphur-containing biocide may be a non-oxidising or oxidising biocide.

Suitably the sulphur-containing biocide comprises a thiocarbonate, a thiocyanate, an isothiocyanate, a dithiocarbonate, a thiazole, a thiazolinone, an isothiazolinone, or any mixture or derivative thereof, but is preferably a thiazolinone or an isothiazolinone.

Suitable isothiazolinones include linear or branched C₁-C₆ alkylhalothiazolinones, or alkylhaloisothiazolinones.

Preferred isothiazolinones include methylispthiozolinones, especially 2-methyl-4-isothiazolin-3-one, and methylhaloisothiazolinones, especially 5-chloro-2-methyl-4-isothiazolin-3-one.

Suitably the sulphur-containing biocide is present in an amount of at least 0.001 wt % of the total weight of the composition, preferably at least 0.0025wt %, and more preferably at least 0.005wt %.

Suitably the sulphur-containing biocide is present in an amount of no more than 0.1wt % of the total weight of the composition, preferably no more than 0.05wt % and more preferably no more than 0.01 wt %.

The sulphur-containing biocide may also contain one or more nitrogen atoms, such as in the case of thiazoles and thiazolinones, and thus in some embodiments of the invention there may be a single biocide which comprises both a nitrogen atom and a sulphur atom but the biocidal composition of the invention preferably comprises both a nitrogen-containing biocide and a separate sulphur containing biocide, whether or not the sulphur-containing biocide comprises nitrogen atoms or not.

The aqueous biocidal composition preferably comprises at least 70wt % water, more preferably at least 80wt % and most preferably at least 90wt %, based on the total weight of the composition.

Suitably the oleophilic carrier comprises an oleophilic solvent such as an oleophilic glycol for example. The oleophilic glycol is suitably a C₁-C₈ linear or branched alkylene glycol or dialkylene glycol. Preferred as a glycol is dipropylene glycol.

Alternatively the oleophilic carrier may comprise one or more natural or synthetic compounds, preferably aromatic compounds, such as natural oils, natural scents and fragrances and their synthesized equivalents for example. The natural compounds may be plant or animal derived.

Particularly suitable natural compounds or oleophilic carriers include aromatic aldehydes and ketones such as amyl cinnamic aldehyde, plant oils such as lavender oil, patchouli oil, peppermint oil and citrus oil, and aromatic alcohols such as citronellol and xyllols for example. Natural oils may be mixed with natural or synthetic gums and/or resins.

Preferably the oleophilic carrier comprises a mixture of natural oils, scents and/or fragrances.

Suitably the oleophilic carrier is present in the composition in an amount of at least 1wt % of the total weight of the composition, preferably at least 1.5wt % and more preferably at least 2wt %.

Suitably the oleophilic carrier is present in the composition in an amount of no more than 8wt % of the total weight of the composition, preferably no more than 6wt % and more preferably no more than 4wt %.

The composition may include further ingredients such as an alcohol, preferably 2-propanol, and an inorganic salt, preferably a magnesium salt such as magnesium chloride or magnesium nitrate for example.

The composition may also include a defoamer, which allows the present invention to be used as a laundry additive.

According to a second aspect of the invention there is provided a method of dispersing a biocide, the method comprising the steps of:
(a) providing a biocidal composition of the first aspect of the invention; and
(b) evaporating at least a portion of the biocidal composition.

Suitably step (b) comprises evaporating the biocidal composition in a substantially enclosed space, such as a room, hospital ward, ship's cabin, gymnasium, or the like, for example.

Suitably step (b) comprises evaporating the biocidal composition at a rate sufficient to maintain a steady concentration of biocidal composition in the atmosphere of the enclosed space at a desired concentration.

Step (b) may comprise continuous evaporation of the biocidal composition or intermittent evaporation.

Evaporation may be effected by heating the biocidal composition, or by heating the atmosphere adjacent to the biocidal composition. Evaporation may be effected by allowing the biocidal composition to evaporate at ambient temperature, such as room temperature for example. Alternatively evaporation may be effected by effecting a substantial airflow across the biocidal composition.

The biocidal composition may be packaged in a container comprising a wick through which the biocidal composition is drawn, and from which the biocidal composition is evaporated. The wick may be located adjacent to a heating element or an airflow effecting means, such as a fan, for example.

According to a third aspect of the invention there is provided a biocidal delivery device comprising a reservoir containing a biocidal composition of the first aspect of the invention, and means to evaporate at least a portion of the biocide.

The evaporation means may comprise a heating element or a means to effect substantial airflow across the biocidal composition, such as a fan, for example. Suitably the device further comprises a wick, connected to the reservoir and arranged to draw up the biocidal composition from the reservoir.

According to a fourth aspect of the invention there is provided a biocidal composition comprising a nitrogen-coating biocide, a sulphur-containing biocide, a glycol, and water.

Suitably the nitrogen-containing biocide is a quaternary ammonium-containing biocide, the sulphur-containing biocide is an isothiazolinone and the glycol is dipropylene glycol.

According to a fifth aspect of the present invention there is provided a biocidal composition comprising a nitrogen-containing biocide, a sulphur-containing biocide, one or more natural or synthetic aromatic compounds, and water.

Suitably the nitrogen-containing biocide is a quaternary ammonia containing biocide, the sulphur-containing biocide is an isothiazolinone and the natural or synthetic aromatic compound is selected from an oil, an aldehyde, a ketone and an aromatic alcohol.

The compositions of the fourth and fifth aspects of the invention provide particularly useful characteristics in biocidal efficiency and: sustainable evaporation rates.

According to a sixth aspect of the present invention there is provided a pouch adapted to locate around and protect produce, the pouch comprising a material incorporated in which is a biocidal composition according to the first, fourth or fifth aspect of the present invention.

The term produce is used to mean fresh produce, such as vegetables and fruit, as they are growing or after harvest. Such a pouch may be used to protect produce on the plant, by being paced over the fruit or vegetable parts and fixed in place. This will prevent or reduce the risk of biological attach from insects or microbes. After harvest the produce may be stored in such a pouch.

The pouch may be substantially wholly formed from the biocide containing material, which may often be flexible sheet material formed round into a pouch shape with a suitable closure. The material may be absorbent and the biocidal composition may be incorporated therein by absorption. The material may instead be a plastics material with the biocidal composition incorporated in that plastics material, either during formation of the pouch from that plastics material or in manufacture of the plastics material itself.

The biocidal composition may also be incorporated into plastics material for the construction of other items that desirably require a biocidal effect.

### EXAMPLES

The various embodiments of the invention will now be described by way of non-limiting example in which the following materials are used:
N,N-didecyl-N-N-dimethyl ammonium chloride—supplied by Lonza. UK.
5-Chloro-2-methyl-4-isothiazolin-3-one—Lonza, UK.
2-Methyl-4-isothiazolin-3-one—Lonza, UK.
Magnesium chloride and magnesium nitrate—Lonza, UK.
Fragrance Composition—a multi-component mixture of natural and synthetic aromatic materials consisting of amyl cinnamic aldehyde, citronellol, coumarin, diphenyl oxide, lavandin grosso, musk xylol and oak moss resin, supplied by Dale Air Limited, Lytham, UK.
Dipropylene glycol—supplied by Dale Air Limited, Lytham UK.

Two formulations, Formulation A and Formulation B were prepared as set out in Table 1 below:

TABLE 1

| | Formulation A | Formulation B |
|---|---|---|
| Ingredient | % w/w | % w/w |
| N,N-Didecyl-N-N- | 2.25 | 2.25 |
| dimethylammonium chloride | | |
| 2-propanol | 0.9 | 0.9 |
| 5-Chloro-2-methyl-4- | 0.005 | 0.005 |
| isothiazolin-3-one | | |
| 2-Methyl-4-isothiozolin-3- | 0.001 | 0.001 |
| one | | |
| Magnesium chloride and | 0.112 | 0.112 |
| magnesium nitrate 50:50 | | |
| Water | 94.232 | 94.232 |
| Dipropylene glycol | 2.5 | 0 |
| Fragrance Composition | 0 | 2.5 |

Thus, the Fragrance Composition in Formulation B and the dipropylene glycol in Formulation A serve as the oleophilic carrier according to the invention. A Control Formulation was also prepared, in which the ingredients were identical to Formulation A and B, with the exception that dipropylene glycol, N,N-Didecyl-N-N-dimethylammonium chloride and each of the isothiazolinones were absent, such that the Control Formulation did not substantially include an oleophilic carrier or biocide of the invention.

In each of Formulations A and B, and the Control Formulation the ingredients were added to the water component of formulations, and thoroughly mixed. In the case of Formulations A and B, thorough mixing was achieved relatively rapidly, and no residual solids were noted. In the case of the Control Example, mixing was difficult, in the absence of an oleophilic carrier, and separation into some of the components was noticed.

Each of the three Formulations (29 g of Formulation A, 30 g of Formulation B and the Control Formulation) were then put into a reservoir in separate plug-in vaporisers, of the sort which comprises a reservoir and a wick, around which is located a heating element as is well known to persons skilled in the art. The plug-in vaporisers were plugged into a domestic electricity socket in separate enclosed rooms, and activated. In each case, biocidal composition was withdrawn through the wick, and evaporated at a steady concentration by application of heat from the heating element in the region around the wick. The vaporisers included diffusion apertures in order to allow evaporated composition out of the vaporisers and into the atmosphere of the enclosed room.

The diffusers were set to run continually for 7 days.

After seven days, the diffusers were turned off, and the amount of remaining Formulation determined. The experiments were repeated a number of times. It was found that between 7 and 11 g of the Control Formulation had evaporated from the diffuser over 7 days, whereas only 2 to 3.5 g of Formulations A to B had evaporated over the identical period.

The experiment was repeated again for a period of 7 days, but in this case every 24 hours the position of the diffuser in the enclosed space was varied, such that a variety of conditions such as heat, immediate air space volume etc was changed every 24 hours. The results of this experiment showed that Formulations A and B had a much slower discharge whatever the ambient conditions than the Control Formulation.

These results indicate that the oleophilic carrier effects reduced evaporation speeds of the biocidal compositions of the invention, which in use, effects slower release rates and conserves wastage of biocidal composition.

In order to test the biocidal efficacy of each of Formulations A, B, and the Control Formulation, a plate containing a colony of green algae was positioned 5 metres away from diffusers containing Formulations A, B and the Control Formulation, each in separate enclosed spaces. The diffusers were left to operate for, a period of 24 hours under ambient conditions. After 24 hours the diffusers were switched off, and the green algae inspected. The results showed that there was a noticeable reduction in the area of the plate covered by green algae in the enclosed spaces in which Formulations A and B had been diffused, but there was no reduction in the amount of green algae on the plate in the enclosed space in which the Control Formulation had been diffused. These experiments were repeated, and in each case, diffusion of Formulation A or B in the enclosed space in which green algae was positioned gave substantial reductions in the amount of green algae present on the plate, whereas the green algae on the plate in which the Control Formulation was diffused gave no reduction in the amount of green algae.

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. An aqueous biocidal composition comprising: a nitrogen-containing biocide, a sulphur-containing biocide and an oleophilic carrier.

2. An aqueous biocidal composition as claimed in claim 1 wherein the nitrogen-containing biocide comprises an amide biocide, a triazine, a guanidine, a quaternary ammonium compound, or mixtures or derivatives thereof.

3. An aqueous biocidal composition as claimed in claim 1 wherein the nitrogen-containing biocide comprises an optionally substituted linear or branched C₁-C₂₀ alkyl ammonium halide or alkylbenzylammonium halide.

4. An aqueous biocidal composition as claimed in claim 3 wherein the alkylammonium halide is a dialkyldimethyl ammonium halide or an alkyldimethylbenzyl ammonium chloride.

5. An aqueous biocidal composition as claimed in claim 1 wherein the nitrogen-containing biocide is present in the composition in an amount of at least 1wt % of the total weight of the composition.

6. An aqueous biocidal composition as claimed in claim 1 wherein the nitrogen-containing biocide is present in the composition in an amount of no more than 10wt % of the total weight of the composition.

7. An aqueous biocidal composition as claimed in claim 1 wherein the sulphur-containing biocide comprises a thiocarbonate, a thiocyanate, an isothiocyanate, a dithiocarbonate, a thiazole, a thiazolinone, an isothiazolinone, or any mixture or derivative thereof.

8. An aqueous biocidal composition as claimed in claim 1 wherein the sulphur-containing biocide comprises methylisothiozolinones, and methylhaloisothiazolinones.

9. An aqueous biocidal composition as claimed in claim 1 wherein the sulphur-containing biocide is present in an amount of at least 0.001wt % of the total weight of the composition.

10. An aqueous biocidal composition as claimed in claim 1 wherein the sulphur-containing biocide is present in an amount of no more than 0.1 wt % of the total weight of the composition.

11. An aqueous biocidal composition as claimed in claim 1 which preferably comprises at least 70wt % water, based on the total weight of the composition.

12. An aqueous biocidal composition as claimed in claim 1 wherein the oleophilic carrier comprises an oleophilic solvent selected from the group consisting of an oleophilic glycol, a C₁-C₈ linear or branched alkylene glycol, and dialkylene glycol.

13. An aqueous biocidal composition as claimed in claim 1, wherein the oleophilic carrier comprises one or more natural or synthetic compounds selected from the groups consisting of aromatic aldehydes and ketones, amyl cinnamic aldehyde, plant oils, lavender oil, patchouli oil, peppermint oil, citrus oil, aromatic alcohols, citronellol and xyllols.

14. An aqueous biocidal composition as claimed in claim 1, wherein the oleophilic carrier comprises a mixture of natural oils, scents or fragrances.

15. An aqueous biocidal composition as claimed in claim 1 wherein the oleophilic carrier is present in the composition in an amount of at least 1 wt % of the total weight of the composition.

16. An aqueous biocidal composition as claimed in claim 1 wherein the oleophilic carrier is present in the composition in an amount of no more than 8wt % of the total weight of the composition.

17. An aqueous biocidal composition as claimed in claim 1 which includes further ingredients selected from an alcohol, a defoamer and an inorganic salt.

18. A method of dispersing a biocide, the method comprising the steps of:
(a) providing a biocidal composition according to claim 1; and
(b) evaporating at least a portion of the biocidal composition.

19. A method of dispersing a biocide as claimed in claim 18, wherein step (b) comprises evaporating the biocidal composition in a substantially enclosed space.

20. A method of dispersing a biocide as claimed in claim 19, wherein step (b) comprises evaporating the biocidal composition at a rate sufficient to maintain a steady concentration of biocidal composition in the atmosphere of the enclosed space at a desired concentration.

21. A method of dispersing a biocide as claimed in claim 18, wherein step (b) comprises continuous evaporation of the biocidal composition or intermittent evaporation.

22. A method of dispersing a biocide as claimed in claim 18, wherein evaporation is effected by heating the biocidal composition, or the atmosphere adjacent to the biocidal composition, or by effecting a substantial airflow across the biocidal composition.

23. A biocidal delivery device comprising a reservoir containing a biocidal composition according to claim 1, and means to evaporate at least a portion of the biocide.

24. A biocidal delivery device as claimed in claim 23, where the evaporation means comprise a heating element or a means to effect substantial airflow across the biocidal composition.

25. A biocidal delivery device as claimed in claim 23, wherein the device further comprises a wick, connected to the reservoir and arranged to draw up the biocidal composition from the reservoir.

26. A biocidal composition comprising a nitrogen-containing biocide, a sulphur-containing biocide, water and a glycol or one or more natural or synthetic aromatic compounds.

27. A biocidal composition as claimed in claim 26, wherein the nitrogen-containing biocide is a quaternary ammonium-containing biocide, the sulphur-containing biocide is an isothiazolinone and the glycol is dipropylene glycol.

28. (cancelled)

29. (cancelled)

30. (cancelled)

31. (cancelled)

32. A biocidal composition according to claim 1, wherein the composition is incorporated in a material adapted to form a pouch.

33. A biocidal composition according to claim 26, wherein the composition is incorporated in a material adapted to form a pouch.
